(19) United States
(12) Patent Application Publication
(10) Pub. No.: US 2022/0084658 A1
(43) Pub. Date: Mar. 17, 2022

(54) Systems and Methods for Automated Vessel Labeling

(71) Applicant: FUJIFILM MEDICAL SYSTEMS U.S.A., INC., Morrisville, NC (US)

(72) Inventors: Richard Adam Hooker, Fishers, IN (US); Greg Hoofnagle, Cary, NC (US); Roger Newcomer, Slipper Rock, PA (US); Michael Manders, Reno, NV (US); David Funabashi, Durham, NC (US); Richard Craig, Lakewood, CO (US)

(21) Appl. No.: 17/024,555

(22) Filed: Sep. 17, 2020

(51) Int. Cl. G16H 30/40; G16H 30/20; G06T 7/70; G06T 7/13; G06T 7/00; G06K 9/32; A61B 6/00

(52) U.S. Cl. CPC G16H 30/40; G16H 30/20; G06T 7/70; G06T 7/13; A61B 6/504; G06K 9/3233; A61B 6/469; G06T 2207/30101; G06T 2207/30092; G06T 7/0012

(57) ABSTRACT

Method for labeling a vessel in a medical image record includes receiving, at one or more computing devices, the medical image record, the medical image record including a camera angle; analyzing, at the one or more computing devices, the medical image record to locate and identify one or more vessels, wherein analyzing the medical image record is based on edge detection, one or more anatomical landmarks in the medical image record, and the camera angle; and displaying, at the one or more computing devices, the medical image record including a label associated with at least one of the one or more vessels.

### BACKGROUND

### 1. Field of Disclosed Subject Matter

The disclosed subject matter is directed to systems and methods for automated vessel labeling. For example, the systems and methods described herein can detect and label vessels in medical images, and more specifically, Digital Imaging and Communications in Medicine (“DICOM”) Objects.

### 2. Description of Related Art

In medical imaging, Picture Archiving and Communication Systems (“PACS”) are a combination of computers and networks dedicated to the storage, retrieval, presentation, and distribution of images. While medical information can be stored in a variety of formats, a common format of image storage is DICOM. DICOM is a standard in which, among other things, medical images and associated meta-data can be communicated from imaging modalities (e.g., x-ray (or x-rays' digital counterparts: computed radiography (“CR”) and digital radiography (“DR”)), X-ray angiography (“XA”), computed tomography (“CT”), and magnetic resonance imaging (“MRI”) apparatuses) to remote storage and/or client devices for viewing and/or other use.

An exemplary medical image that can be stored in DICOM is an angiogram. Angiography is a technique that includes delivering an x-ray opaque dye (called a contrast or contrast agent) to vessels in the body and then utilizing x-ray imaging to view the dye flowing within the vessels to identify the shape, size, and/or condition of the vessels. Angiography can be used to diagnose a variety of issues in blood vessels, such as blockages, vessel narrowing, or aneurysms.

Professionals viewing medical images often require presentation of data associated with a patient, beyond merely the medical image. Furthermore, professionals can seek features that can make review quicker and more efficient. For example, when viewing an angiogram of a plurality of blood vessels, it may be helpful to present the professionals with the identity of the blood vessels in the image, and additional information, such as prosthetic devices (e.g., stents) previously placed within the blood vessels.

Accordingly, there is a need for systems and methods for automated vessel labeling.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended figures.

To achieve these and other advantages and in accordance with the purpose of the disclosed subject matter, as embodied and broadly described, the disclosed subject matter is directed to systems and methods for labeling a vessel in a medical image record. For example, a method for labeling a vessel in a medical image record includes receiving, at one or more computing devices, the medical image record, the medical image record including a camera angle; analyzing, at the one or more computing devices, the medical image record to locate and identify one or more vessels, wherein analyzing the medical image record is based on edge detection, one or more anatomical landmarks in the medical image record, and the camera angle; and displaying, at the one or more computing devices, the medical image record including a label associated with at least one of the one or more vessels.

The medical image record can be a DICOM Service-Object Pair Instance. The medical image record and label can be a DICOM Secondary Capture Image Information Objection Definition.

The vessel can be one of a blood vessel, a bile duct, and a ureter. The method can include identifying a region of interest associated with at least one of the one or more vessels. The method can include labeling the region of interest. The method can include retrieving additional information related to the region of interest.

In accordance with the disclosed subject matter, one or more computer-readable non-transitory storage media embodying software are provided. The software can be operable when executed to: receive, at one or more computing devices, a medical image record, the medical image record including a camera angle; analyze, at the one or more computing devices, the medical image record to locate and identify one or more vessels, wherein analyzing the medical image record is based on edge detection, one or more anatomical landmarks in the medical image record, and the camera angle; and display, at the one or more computing devices, the medical image record including a label associated with at least one of the one or more vessels.

In accordance with the disclosed subject matter, a system including one or more processors; and a memory coupled to the processors including instructions executable by the processors are provided. The processors can be operable when executing the instructions to: receive, at one or more computing devices, a medical image record, the medical image record including a camera angle; analyze, at the one or more computing devices, the medical image record to locate and identify one or more vessels, wherein analyzing the medical image record is based on edge detection, one or more anatomical landmarks in the medical image record, and the camera angle; and display, at the one or more computing devices, the medical image record including a label associated with at least one of the one or more vessels.

### DRAWINGS

FIG. 1 shows a hierarchy of medical image records that can be compressed and stored in accordance with the disclosed subject matter.

FIG. 2 shows the architecture of a system for labeling a vessel in a medical image record, in accordance with the disclosed subject matter.

FIGS. 3A-E illustrate medical image records including and not including labels, in accordance with the disclosed subject matter.

FIG. 4 is a flow chart of a method for labeling a vessel in a medical image record in accordance with the disclosed subject matter.

### DETAILED DESCRIPTION

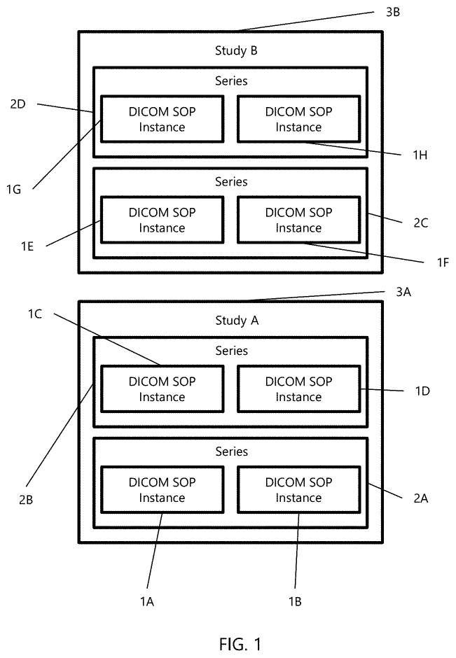

Reference will now be made in detail to various exemplary embodiments of the disclosed subject matter, exemplary embodiments of which are illustrated in the accompanying figures. For purpose of illustration and not limitation, the systems and methods are described herein with respect labeling blood vessels, however, the methods and systems described herein can be used for labeling any vessels in a human or animal body, including but not limited to bile ducts and ureters, as well as identifying vessel location and structure. The disclosed methods and systems can also be used to analyze bone structure, for example, to identify a bone fracture, break, or perform vertebral analysis. As used in the description and the appended claims, the singular forms, such as “a,” “an,” “the,” and singular nouns, are intended to include the plural forms as well, unless the context clearly indicates otherwise. Accordingly, as used herein, the term medical image record can refer to one medical image record, or a plurality of medical image records. For example, and with reference to FIG. 1 for purpose of illustration and not limitation, as referred to herein a medical image record can include a single DICOM SOP Instance (also referred to as “DICOM Instance” and “DICOM image”) 1 (e.g., 1A-1H), one or more DICOM SOP Instances 1 (e.g., 1A-1H) in one or more Series 2 (e.g., 2A-D), one or more Series 2 (e.g., 2A-D) in one or more Studies 3 (e.g., 3A, 3B), and one or more Studies 3 (e.g., 3A, 3B). The methods and systems described herein can be used with medical image records stored on PACS, however, a variety of records are suitable for the present disclosure and records can be stored in any system, for example a Vendor Neutral Archive (“VNA”). The disclosed systems and methods can be performed in an automated fashion (i.e., no user input once the method is initiated) or in a semi-automated fashion (i.e., with some user input once the method is initiated).

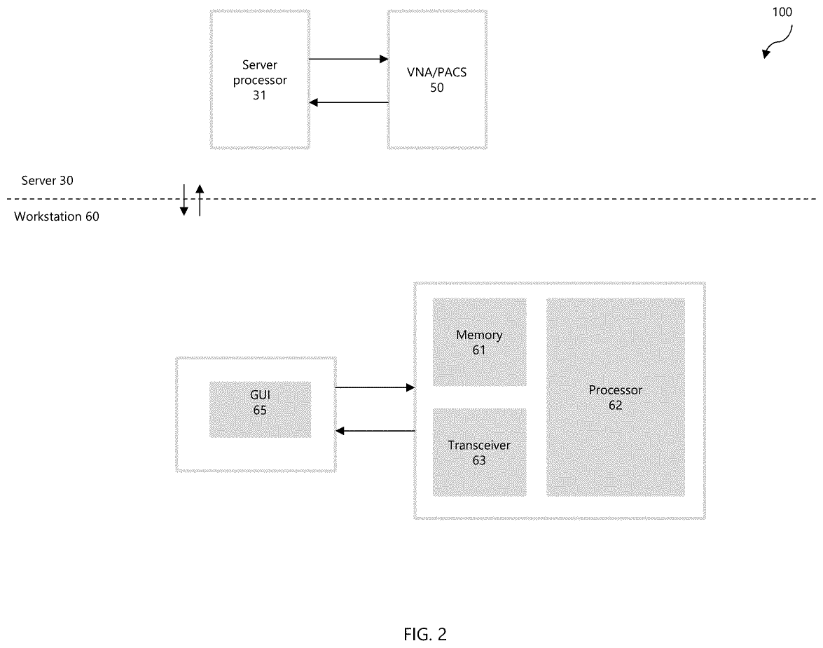

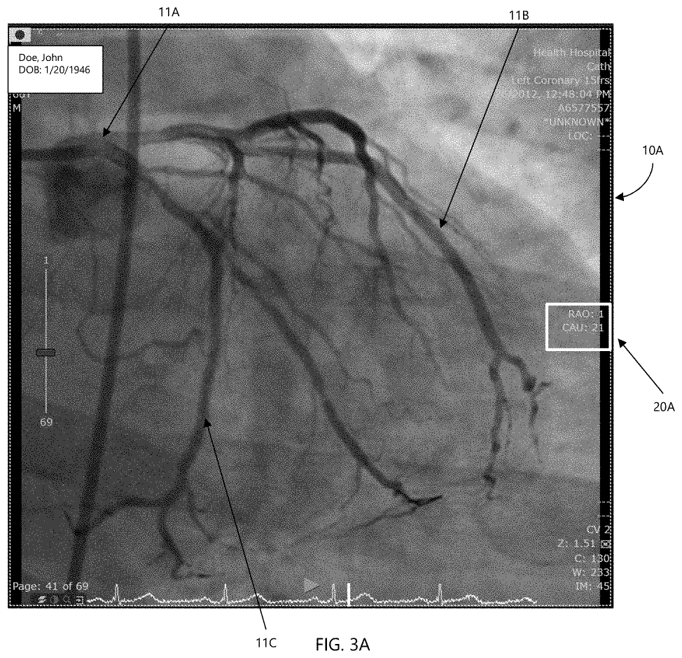

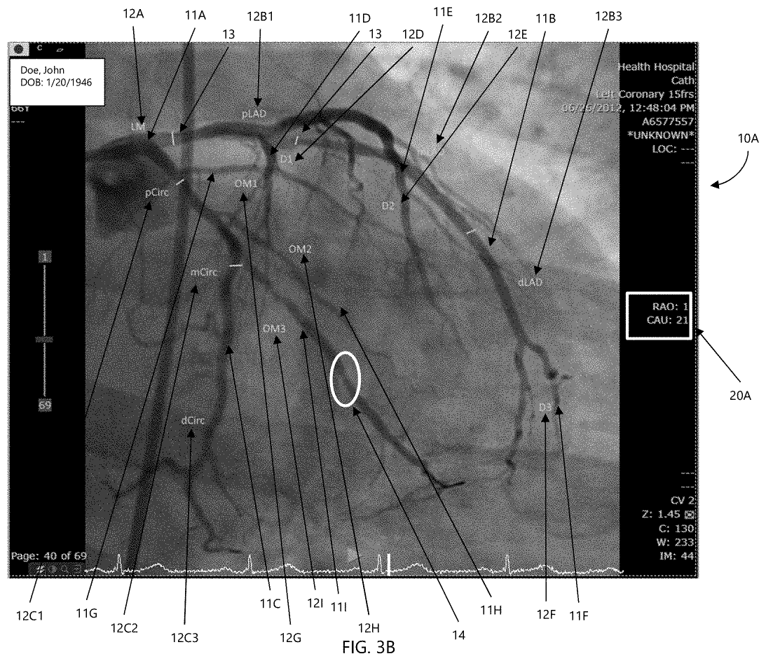

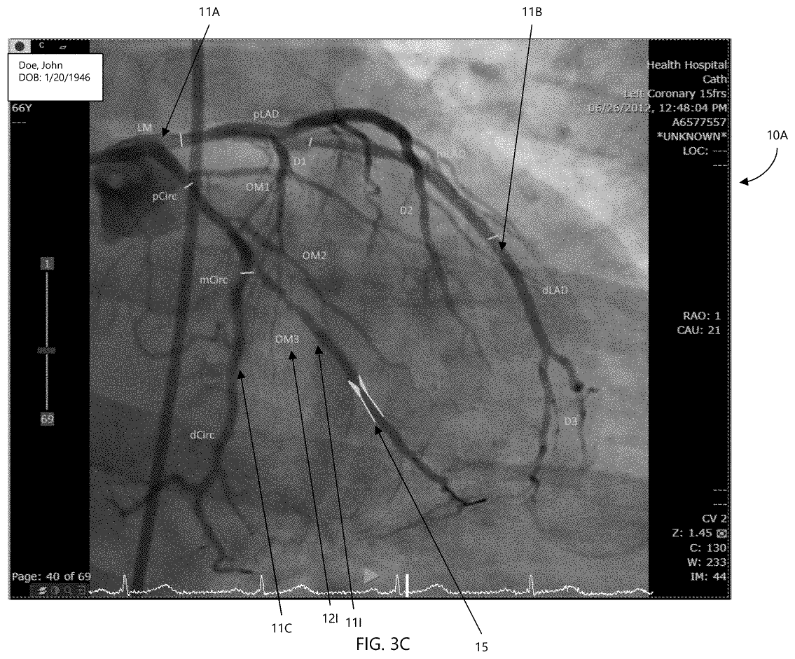

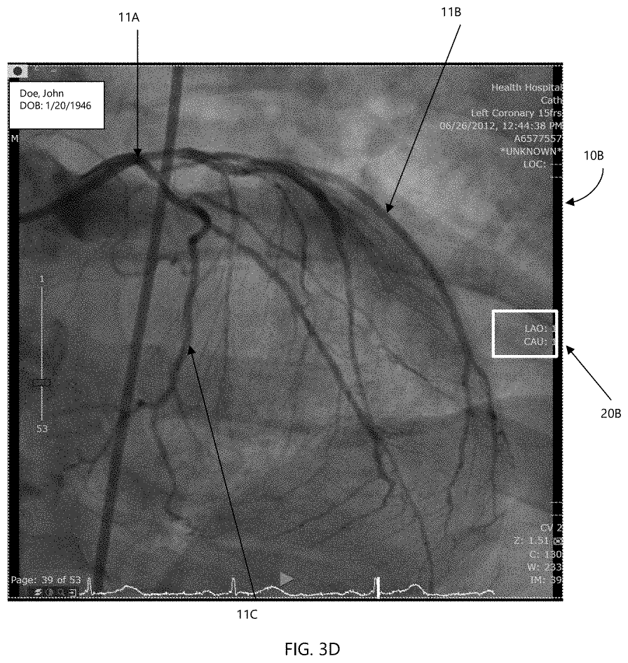

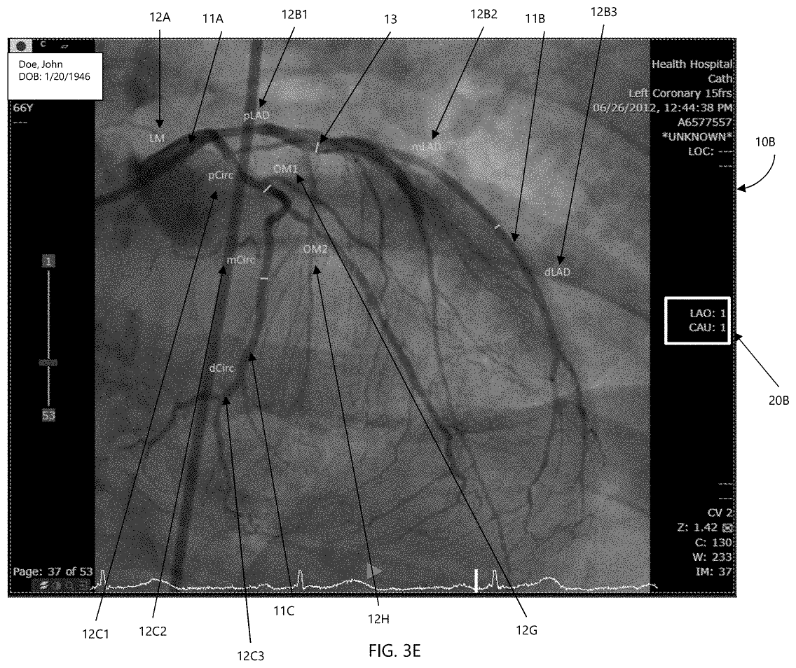

Referring to FIGS. 2-3E for purpose of illustration and not limitation, the disclosed system 100 can be configured to detect and quantify blood vessels 11 (e.g., 11A-11I), as well as display medical image records 10 (e.g., 10A-10B) with the relevant information, for example, included as labels 12 (e.g., 12A-12I). The system 100 can include one or more computing devices defining a server 30 and user workstation 60. The user workstation 60 can be coupled to the server 30 by a network. The network, for example, can be a Local Area Network (“LAN”), a Wireless LAN (“WLAN”), a virtual private network (“VPN”), or any other network that allows for any radio frequency or wireless type connection. For example, other radio frequency or wireless connections can include, but are not limited to, one or more network access technologies, such as Global System for Mobile communication (“GSM”), Universal Mobile Telecommunications System (“UMTS”), General Packet Radio Services (“GPRS”), Enhanced Data GSM Environment (“EDGE”), Third Generation Partnership Project (“3GPP”) Technology, including Long Term Evolution (“LTE”), LTE-Advanced, 3G technology, Internet of Things (“IOT”), fifth generation (“5G”), or new radio (“NR”) technology. Other examples can include Wideband Code Division Multiple Access (“WCDMA”), Bluetooth, IEEE 802.11b/g/n, or any other 802.11 protocol, or any other wired or wireless connection.

Workstation 60 can take the form of any known client device. For example, workstation 60 can be a computer, such as a laptop or desktop computer, a personal data or digital assistant (“PDA”), or any other user equipment or tablet, such as a mobile device or mobile portable media player. Server 30 can be a service point which provides processing, database, and communication facilities. For example, the server 30 can include dedicated rack-mounted servers, desktop computers, laptop computers, set top boxes, integrated devices combining various features, such as two or more features of the foregoing devices, or the like. Server 30 can vary widely in configuration or capabilities, but can include one or more processors, memory, and/or transceivers. Server 30 can also include one or more mass storage devices, one or more power supplies, one or more wired or wireless network interfaces, one or more input/output interfaces, and/or one or more operating systems. Server 30 can include additional data storage such as VNA/PACS 50, remote PACS, VNA, or other vendor PACS/VNA.

A user can be any person authorized to access workstation 60 and/or server 30, including a health professional, medical technician, researcher, or patient. In some embodiments a user authorized to use the workstation 60 and/or communicate with the server 30 can have a username and/or password that can be used to login or access workstation 60 and/or server 30.

Workstation 60 can include GUI 65, memory 61, processor 62, and transceiver 63. Medical image records 10 (e.g., 10A-10B) received by workstation 60 can be processed using one or more processors 62. Processor 62 can be any hardware or software used to execute computer program instructions. These computer program instructions can be provided to a processor of a general purpose computer to alter its function to a special purpose computer, application-specific integrated circuit (“ASIC”), or other programmable digital data processing apparatus, such that the instructions, which execute via the processor of the workstation 60 or other programmable data processing apparatus, implement the functions/acts specified in the block diagrams or operational block or blocks, thereby transforming their functionality in accordance with embodiments herein. The processor 62 can be a portable embedded micro-controller or micro-computer. For example, processor 62 can be embodied by any computational or data processing device, such as a central processing unit (“CPU”), digital signal processor (“DSP”), ASIC, programmable logic devices (“PLDs”), field programmable gate arrays (“FPGAs”), digitally enhanced circuits, or comparable device or a combination thereof. The processor 62 can be implemented as a single controller, or a plurality of controllers or processors.

Workstation 60 can send and receive medical image records 10 (e.g., 10A-10B) from server 30 using transceiver 63. Transceiver 63 can, independently, be a transmitter, a receiver, or both a transmitter and a receiver, or a unit or device that can be configured both for transmission and reception. In other words, transceiver 63 can include any hardware or software that allows workstation 60 to communicate with server 30. Transceiver 63 can be either a wired or a wireless transceiver. When wireless, the transceiver 63 can be implemented as a remote radio head which is not located in the device itself, but in a mast. While FIG. 2 only illustrates a single transceiver 63, workstation 60 can include one or more transceivers 63. Memory 61 can be a non-volatile storage medium or any other suitable storage device, such as a non-transitory computer-readable medium or storage medium. For example, memory 61 can be a random-access memory (“RAM”), read-only memory (“ROM”), hard disk drive (“HDD”), erasable programmable read-only memory (“EPROM”), electrically erasable programmable read-only memory (“EEPROM”), flash memory or other solid-state memory technology. Memory 61 can also be a compact disc read-only optical memory (“CD-ROM”), digital versatile disc (“DVD”), any other optical storage, magnetic cassettes, magnetic tape, magnetic disk storage or other magnetic storage devices, or any other physical or material medium which can be used to tangibly store the desired information or data or instructions and which can be accessed by a computer or processor. Memory 61 can be either removable or non-removable.

Server 30 can include a server processor 31 and VNA/PACS 50. The server processor 31 can be any hardware or software used to execute computer program instructions. These computer program instructions can be provided to a processor of a general purpose computer to alter its function to a special purpose, a special purpose computer, ASIC, or other programmable digital data processing apparatus, such that the instructions, which execute via the processor of the client station or other programmable data processing apparatus, implement the functions/acts specified in the block diagrams or operational block or blocks, thereby transforming their functionality in accordance with embodiments herein. In accordance with the disclosed subject matter, the server processor 31 can be a portable embedded micro-controller or micro-computer. For example, server processor 31 can be embodied by any computational or data processing device, such as a CPU, DSP, ASIC, PLDs, FPGAs, digitally enhanced circuits, or comparable device or a combination thereof. The server processor 31 can be implemented as a single controller, or a plurality of controllers or processors.

As shown in FIGS. 3A-3E medical image records 10 (e.g., 10A-10B) can be an angiogram of a heart and can include a plurality of blood vessels 11 (e.g., 11A-11I) visible due to an x-ray opaque dye flushed into the blood vessels 11 (e.g., 11A-11I) of the heart. For example, medical image records 10A and 10B include dye flushed into the blood vessels 11A-11I of the left coronary tree, and therefore the blood vessels 11A-11I of the left coronary tree are visible in the medical image records 10A-10B. The left coronary tree includes blood vessel 11A which is the left main coronary artery. The left main coronary artery 11A branches into blood vessel 11B, the left anterior descending artery, and blood vessel 11C, the left circumflex artery. The left anterior descending artery 11B can have additional branches extending therefrom, called diagonal branches 11D-11G. Likewise, the left circumflex artery 11C can have additional branches extending therefrom, called obtuse marginal branches 11G-11I. Abbreviations for the names of the blood vessel 11 (e.g., 11A-11I) and portions of blood vessels 11 (e.g., 11A-11I) are summarized in the chart below:

| Vessel name | Abbreviation |
|---|---|
| Left main coronary artery (11A) | LM |
| Left anterior descending artery (11B) | LAD |
| Proximal portion of left anterior artery (11B) | pLAD |
| Middle portion of left anterior artery (11B) | mLAD |
| Distal portion of left anterior artery (11B) | dLAD |
| First diagonal branch (11D) | D1 |
| Second diagonal branch (11E) | D2 |
| Third diagonal branch (11F) | D3 |
| Left circumflex artery (11C) | Circ |
| Proximal portion of left circumflex artery (11C) | pCirc |
| Middle portion of left circumflex artery (11C) | mCirc |
| Distal portion of left circumflex artery (11C) | dCirc |
| First obtuse marginal branch (11G) | OM1 |
| Second obtuse marginal branch (11H) | OM2 |
| Third obtuse marginal branch (11I) | OM3 |

In operation, system 100 can be used to detect and quantify blood vessels 11 (e.g., 11A-11I), as well as display medical image records 10 (e.g., 10A-10B) with the relevant information, for example, included as labels 12 (e.g., 12A-12I). For purpose of illustration and not limitation, FIGS. 3A and 3D show medical image records 10 (e.g., 10A-10B) without labels 12 (e.g., 12A-12I), and FIGS. 3B, 3C, and 3E show medical image records 10 (e.g., 10A-10B) with labels 12 (E.g., 12A-12I). For example, medical image record 10A can be received by workstation 60. Workstation 60 can detect blood vessels 11 (e.g., 11A-11I). For example, Workstation 60 can use an edge detection algorithm.

Workstation 60 can further identify the blood vessels 11 (e.g., 11A-11I). For example workstation 60 can identify that medical image record 10A includes the left main coronary artery 11A, left anterior descending artery 11B, left circumflex artery 11C, and related branches 11D-11H. The workstation 60 can identify portions of certain vessels 11 (e.g., 11A-11I), such as proximal, middle and distal portions of each of the left anterior descending artery 11B and the left circumflex artery 11C. Identifying blood vessels 11 (e.g., 11A-11I) can be performed based on one or more of an edge detection algorithm, anatomical landmarks, and camera angle of the medical image record 10A. Anatomical landmarks can include one or more of bones, vessel location, arrangement, length, and angles. For cardiac angiogram medical images 10 (e.g., 10A-10B), camera angle can refer to, for example, the amount of cranial or caudal angulation (i.e., the amount of rotation of the camera toward the patient's head or feet) and the amount of left anterior oblique (“LAO”) or right anterior oblique (“RAO”) angulation (i.e., rotation of the camera to the patient's left or right). The amount of cranial or caudal angulation and LAO or RAO angulation can be stored within the DICOM file of the medical image 10 (e.g., 10A-10B). For example, box 20A in medical image 10A indicates that the image includes 1 degree of RAO angulation and 21 degrees of caudal angulation. Box 20B (FIG. 3C) in medical image 10B indicates that the image includes 1 degree of LAO angulation and 1 degree of caudal angulation.

Workstation can display the medical image record 10 (e.g., 10A-10B) with labels 12 (e.g., 12A-12I) associated with the vessels 11 (e.g., 11A-11I) or portions of the vessel 11 (e.g., 11A-11I), for example, the abbreviations for the vessels 11 (e.g., 11A-11I) or portions of the vessels 11 (e.g., 11A-11I) as set forth above. For example, the left main coronary artery 11A can be associated with label 12A “LM.” The left anterior descending artery 11B can be associated with label 12B1 “pLAD” for the proximal portion of the left anterior descending artery 11B, label 12B2 “mLAD” for the middle portion of the left anterior descending artery 11B, and label 12B3 “dLAD” for the distal portion of the left anterior descending artery 11B. The diagonal branches 11D-11F extending from the left anterior descending artery 11B can be associated with respective labels 12D-12F. The left circumflex artery 11C can be associated with label 12C1 “pCirc” for the proximal portion of the left circumflex artery 11C, label 12C2 “mCirc” for the middle portion of the left circumflex artery 11C, and label 12C3 “dCirc” for the distal portion of the left circumflex artery. The obtuse marginal branches 11G-11I extending from the left circumflex artery 11C can be associated with respective labels 12G-12I. Divider marks 13 can be provided to indicate where portions of a vessel 11 (e.g., 11B, 11C) start and stop. Likewise FIG. 3C shows medical image record 10B with vessels 11 (e.g., 11A-11C) and labels 12 (e.g., 12A-12C3, 12H, 12G). In accordance with the disclosed subject matter, labels 12 (e.g., 12A-12I) can be added or removed and settings can be adjusted to determine a number of labels 12 (e.g., 12A-12I) displayed.

The system 100 can identify regions of interest (“ROIs”) 14 in the medical images records 10 (e.g., 10A-10B). An ROI 14 can relate to a pathology in a vessel 11 (e.g., 11A-11I). For example, the ROI 14 can be a blood clot, stenosis, occlusion, aneurism, stroke, kidney stone, bile stone or other pathology. The ROI 14 can be identified based on a change in shape in the vessel 11 (e.g., 11A-11I) or an abrupt stop in contrast material (e.g., indicating that no contrast is flowing past a certain point and that the vessel 11 (e.g., 11A-11I) is blocked). Identifying a ROI 14 can be performed based on one or more of an edge detection algorithm, anatomical landmarks, and camera angle of the medical image record 10A. Additionally or alternatively, identifying a ROI 14 can be performed based on additional information retrieved by the system 100, for example, third-party information. The additional information can include hemodynamic system data, vessel segment quantified data, vessel width, vessel dominance, lesion detection, percent stenosis, lesion length, calcification, presences of a graft, stent, or collaterals, inventory used, previously attempts performed (e.g., stents or balloons). For example, ROI 14 can be identified as a lesion in the third obtuse marginal branch 11I, which has label 12I “OM3.” As shown in FIG. 3B, the ROI can be labeled and or annotated, for example, with marker 15. The system 100 can retrieve additional information to help identify a ROI 14. Additionally or alternatively, the system 100 can retrieve additional information regarding ROI 14 after identifying ROI 14.

In accordance with the disclosed subject matter, the labels 12 (e.g., 12A-12I) and markers 15 can be saved and/or exported as DICOM Secondary Capture objects. The disclosed systems and methods can be performed on still medical image records 10 (e.g., 10A-10B) or on live videos.

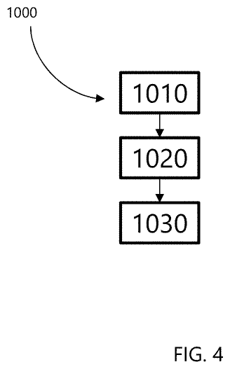

FIG. 4 illustrates an example method 1000 for labeling a vessel in a medical image record. The method 1000 can begin at step 1010, where the method includes receiving, at one or more computing devices, the medical image record, the medical image record including a camera angle. At step 1020 the method can include analyzing, at the one or more computing devices, the medical image record to locate and identify one or more vessels, wherein analyzing the medical image record is based on edge detection, one or more anatomical landmarks in the medical image record, and the camera angle. At step 1030 the method can include displaying, at the one or more computing devices, the medical image record including a label associated with at least one of the one or more vessels. In accordance with the disclosed subject matter, the method can repeat one or more steps of the method of FIG. 4, where appropriate. Although this disclosure describes and illustrates particular steps of the method of FIG. 4 as occurring in a particular order, this disclosure contemplates any suitable steps of the method of FIG. 4 occurring in any suitable order. Moreover, although this disclosure describes and illustrates an example method for labeling a vessel in a medical image record including the particular steps of the method of FIG. 4, this disclosure contemplates any suitable method for labeling a vessel in a medical image record including any suitable steps, which can include all, some, or none of the steps of the method of FIG. 4, where appropriate. Furthermore, although this disclosure describes and illustrates particular components, devices, or systems carrying out particular steps of the method of FIG. 4, this disclosure contemplates any suitable combination of any suitable components, devices, or systems carrying out any suitable steps of the method of FIG. 4.

As described above in connection with certain embodiments, certain components, e.g., server 30 and workstation 60, can include a computer or computers, processor, network, mobile device, cluster, or other hardware to perform various functions. Moreover, certain elements of the disclosed subject matter can be embodied in computer readable code which can be stored on computer readable media and which when executed can cause a processor to perform certain functions described herein. In these embodiments, the computer and/or other hardware play a significant role in permitting the system and method for displaying medical image records. For example, the presence of the computers, processors, memory, storage, and networking hardware provides the ability to display medical image records in a more efficient manner. Moreover, storing and saving the digital records cannot be accomplished with pen or paper, as such information is received over a network in electronic form.

The subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium also can be, or may be included in, one or more separate physical components or media (e.g., multiple CDs, disks, or other storage devices).

The term processor encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA or an ASIC. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC.

Processors suitable for the execution of a computer program can include, by way of example and not by way of limitation, both general and special purpose microprocessors. Devices suitable for storing computer program instructions and data can include all forms of non-volatile memory, media and memory devices, including by way of example but not by way of limitation, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Additionally, as described above in connection with certain embodiments, certain components can communicate with certain other components, for example via a network, e.g., a local area network or the internet. To the extent not expressly stated above, the disclosed subject matter is intended to encompass both sides of each transaction, including transmitting and receiving. One of ordinary skill in the art will readily understand that with regard to the features described above, if one component transmits, sends, or otherwise makes available to another component, the other component will receive or acquire, whether expressly stated or not.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1) A method of labeling a vessel in a medical image record, comprising:
receiving, at one or more computing devices, the medical image record, the medical image record including a camera angle;
analyzing, at the one or more computing devices, the medical image record to locate and identify one or more vessels, wherein analyzing the medical image record is based on edge detection, one or more anatomical landmarks in the medical image record, and the camera angle; and
displaying, at the one or more computing devices, the medical image record including a label associated with at least one of the one or more vessels.

2) The method of claim 1, wherein the medical image record comprises a Digital Imaging and Communications in Medicine (“DICOM”) Service-Object Pair Instance.

3) The method of claim 2, wherein the medical image record and label comprise a DICOM Secondary Capture Image Information Objection Definition.

4) The method of claim 1, wherein the vessel is one of a blood vessel, a bile duct, and a ureter.

5) The method of claim 1, further comprising identifying a region of interest associated with at least one of the one or more vessels.

6) The method of claim 5, further comprising labeling the region of interest.

7) The method of claim 5, further comprising retrieving additional information related to the region of interest.

8) One or more computer readable non-transitory storage media embodying software that is operable when executed to:
receive, at one or more computing devices, a medical image record, the medical image record including a camera angle;
analyze, at the one or more computing devices, the medical image record to locate and identify one or more vessels, wherein analyzing the medical image record is based on edge detection, one or more anatomical landmarks in the medical image record, and the camera angle; and
display, at the one or more computing devices, the medical image record including a label associated with at least one of the one or more vessels.

9) The media of claim 8, wherein the medical image record comprises a Digital Imaging and Communications in Medicine (“DICOM”) Service-Object Pair Instance.

10) The media of claim 9, wherein the medical image record and label comprise a DICOM Secondary Capture Image Information Objection Definition.

11) The media of claim 8, wherein the vessel is one of a blood vessel, a bile duct, and a ureter.

12) The media of claim 8, wherein the software is further operable to identify a region of interest associated with at least one of the one or more vessels.

13) The media of claim 12, wherein the software is further operable to label the region of interest.

14) The media of claim 12, wherein the software is further operable to retrieve additional information related to the region of interest.

15) A system comprising: one or more processors; and a memory coupled to the processors comprising instructions executable by the processors, the processors being operable when executing the instructions to:
receive, at one or more computing devices, a medical image record, the medical image record including a camera angle;
analyze, at the one or more computing devices, the medical image record to locate and identify one or more vessels, wherein analyzing the medical image record is based on edge detection, one or more anatomical landmarks in the medical image record, and the camera angle; and
display, at the one or more computing devices, the medical image record including a label associated with at least one of the one or more vessels.

16) The system of claim 15, wherein the medical image record comprises a Digital Imaging and Communications in Medicine (“DICOM”) Service-Object Pair Instance.

17) The system of claim 16, wherein the medical image record and label comprise a DICOM Secondary Capture Image Information Objection Definition.

18) The system of claim 15, wherein the vessel is one of a blood vessel, a bile duct, and a ureter.

19) The system of claim 15, wherein the processors are further operable to identify a region of interest associated with at least one of the one or more vessels.

20) The system of claim 19, wherein the processors are further operable to label the region of interest.

21) The system of claim 19, wherein the processors are further operable to retrieve additional information related to the region of interest.
